SEQUENCE LISTING (9 sequences)

SEQ ID NO: 1 - DNA, 4957 bp, Synechococcus sp.
aagcttggattgaagcgatcggggtcaatcccagcgatgatcctcagttcctcctgatggtcgatccctttagcgccaagattgaggatctgctgcaagggctggatttcgcctatcccgaggccgtgaaagtgggcggattggccagtggtttgggggcagagtcagcgatcgccagcttgttttttcaagaccgacaggtcgatggcgtgattgggctagccctcagtggcaatgtccagctgcaggcgatcgtggctcagggctgtcgtccagttggcccgctttggcatgtggcagcggcggagcgcaacattctgcggcaacttcagaccgaagacgaggaaccgatcgccgcgctgcaagccctacagtcagtcctgcgtgatctctcccctgaattacagcgatcgctctgtgtgggcctggcctgcaattctttccaaacggtattacaaccgggcgacttcctgatccgtaacctgctggggtttgatccccgcactggtgctgtagcaatcggcgatcgcattcgagttgggcagcggctgcagctgcacgtacgggatgcccagacagcggcggatgacctcgagcggcaactggggcaatggtgccggcagcatgcgacaaaaccagcagcttccctcttgttttcctgcttggggcgcggcaagcccttctatcagcaggccaacttcgagtcgcaactgattcagcattacctctcagagctgcccctagctggctttttctgtaatggcgaaatcggcccgatcgctggcagcacctacctgcatggctacacatcggtgctggctttgctgtcggccaaaactcactagcgccagcgagacctgattgtcgatctgctgagcgcgactgtagcgctggaaataggcccggacctgagcaggcgcatcggccaagctgaccgtagtatcaccgtcagccacccccgcccagaaattccgcaacatcggcaggagagcgatcgcctccgcctccgataaattcaacggctcatgggtcaacaggcggatcaagtactctgactgcgatcgccatccattcccgccgaaaacgtttgtaaatcagtcttgatccggtagcgatcgcacccgacgggactctagttctagttgccaaccttcagcggcaggttgtacggttccgagtcggtagggatggggatagctgaccaaggaaccggtcgtgacttcccagagagcaccttgctgactggtggcttggatgtggaggtggcctgtgaagatcaccgagacgctgcccgcttcgaggattgatcgcaattcctcggcattttctaagatgtagcgctgaccaagcggatgctgctgttgatcgggcagatgctccaacacattgtggtgaatcatcacccagcgttggctagcggtggaagtggcgagttcttgttgcagccagttgagttgcgcgcaatcgactcgcccccgatgcagttgatggcccgcttcatcaaaagcgatcgaattcagcgcaaacagatcgagatccggtgcgatcgtgcagcgatagtaggggcgatcgctcgtgaagccaaagtcttgatagagctcgacaaactcggccacaccggtgcgatcgcgatcgctcgctgcggcgggcatatcgtggttgcccggcaccacatagaccggatagggcaactggcgcaattgttgcagcagccactgatggttttcccgctccccgtgctgggttaaatcccccggcagcaacaggaagtccaaatccagcgctgccagttctgtcaggatttgctcaaaagccggaatgctgcactcaatcaaatggaagcgatggggatggtgccaaattgtctgcggcagtccaatgtggagatcgctcagcagcgcaaatcgaaacgctcggttcattgccatcccctcagctatcgagcccgattctaggcgaagctaggtcgagtccgttgtcttcagttgcaagcattcatggccagagttcgcgttcggcagcacgtcaatccgctctctcagaaattccaagtggtcacgacttggccggattggcaacaggtctatgcggactgcgatcgcccgctgcatttggatattggctgtgctcgcgggcgctttctgctggcaatggcgacacgacaacctgagtggaattatctggggctggaaattcgtgagccgctggtagatgaggcgaacgcgatcgcccgcgaacgtgaactgaccaatctctactaccacttcagcaacgccaatttggacttggaaccgctgctgcgatcgctgccgacagggattttgcagcgggtcagcattcagttcccggatccttggttcaagaaacgccatcaaaagcgacgcgtcgtccagccggaactggtgcaagccctcgcgactgcgttacctgctggtgcagaggtctttctgcaatccgatgtgctggaagtgcaggcagagatgtgcgaacactttgcggcggaaccccgctttcagcgcacctgcttggactggctgccggaaaatccgctgcccgtcccgaccgagcgcgaaattgccgttcaaaacaaacagttgccagtctaccgtgctctcttcattcggcagccagcggactaagctcttaaggcaagcgttgacgcgatcgcgatgactgtctggcaaactctgacttttgcccattaccaaccccaacagtggggccacagcagtttcttgcatcggctgtttggcagcctgcgagcttggcgggcctccagccagctgttggtttggtctgaggcactgggtggcttcttgcttgctgtcgtctacggttcggctccgtttgtgcccagttccgccctagggttggggctagccgcgatcgcggcctattgggccctgctctcgctgacagatatcgatctgcggcaagcaacccccattcactggctggtgctgctctactggggcgtcgatgccctagcaacgggactctcacccgtacgcgctgcagctttagttgggctagccaaactgacgctctacctgttggtttttgccctagcggctcgggttctccgcaatccccgtctgcgatcgctgctgttctcggtcgtcgtgatcacatcgctttttgtcagtgtctacggcctcaaccaatggatctacggcgttgaagagctggcgacttgggtggatcgcaactcggttgccgacttcacctcacgggtttacagctatctgggcaaccccaacctgctggctgcttatctggtgccgacgactgccttttctgcagcagcgatcggggtgtggcgcggctggctccccaagctgctggcgatcgctgcgacaggtgcgagcagcttatgtctgatcctcacctacagtcgcggtggctggctgggttttgtcgccatgatttttgtctgggcgttattagggctctactggtttcaaccccgtctacccgcaccctggcgacgctggctattcccagtcgtattgggtggactagtcgcggtgctcttggtggcggtgcttggacttgagccgttgcgcgtgcgcgtgttgagcatctttgtggggcgtgaagacagcagcaacaacttccggatcaatgtctggctggcggtgctgcagatgattcaagatcggccttggctgggcatcggccccggcaataccgcctttaacctggtttatcccctctatcaacaggcgcgctttacggcgttgagcgcctactccgtcccgctggaagtcgcggttgagggcggactactgggcttgacggccttcgcttggctgctgctggtcacggcggtgacggcggtgcggcaggtgagccgactgcggcgcgatcgcaatccccaagccttttggttgatggctagcttggccggtttggcaggaatgctgggtcacggtctgtttgataccgtgctctatcgaccggaagccagtacgctctggtggctctgtattggagcgatcgcgagtttctggcagccccaaccttccaagcaactccctccagaagccgagcattcagacgaaaaaatgtagcgggctccccaacaaattcctgtgcacccgactggatccaccacctaaactggatcccaaaggtatccggtggatctagggtcataacgaactccgaccgcgatcgcgtccgcgaactgaacctccatcgcaccgaagcggagttcgttagtcgttgaagagccaatgctagagggggctgccgaagcagttgggctggaagcaggctgcgagaagccacccgcatccaaggcaaagttcagccgaccttccgcaaagactacgatcgccacggcggctctgccagctaagtcagcgctgggttagttgtcatagcagtccgcagacaagttaggacaacttcatagagggactcgctcagagtcaacagccgctgtccgtgggggtgcgcaatcacccccacacccacgcactgggggactcgactcccccaggccccccgcaacaagatttcggataaggggcatcggctgaatcgcgatcgctgcgggtaaaactagccggtgttagccatgggtttgagactaatcggcacggggcaaaacgtcctgatttatttgctcaatgtgataggttacatcgtcaaaaacaaggcccaagaggtaggaaaaatcacgaccgcccaagtccgagggctttgctgttgggagcgacctagggcagactagacagagcattgctgtgagccaaagcgccttcaattgctggcggctgtgggtttttcggaggttgccaaatgaaagaccttttcgtcaatgtcctccgctatccccgctacttcatcaccttccagctgggtattttttagtcgatctaccagtgggtgcggccgatggttcgcaacccagtcgcggcttgggcgctgctaggctttggagtttcga

SEQ ID NO: 2 - DNA, 1404 bp, Synechococcus sp.
atgactgtctggcaaactctgacttttgcccattaccaaccccaacagtggggccacagcagtttcttgcatcggctgtttggcagcctgcgagcttggcgggcctccagccagctgttggtttggtctgaggcactgggtggcttcttgcttgctgtcgtctacggttcggctccgtttgtgcccagttccgccctagggttggggctagccgcgatcgcggcctattgggccctgctctcgctgacagatatcgatctgcggcaagcaacccccattcactggctggtgctgctctactggggcgtcgatgccctagcaacgggactctcacccgtacgcgctgcagctttagttgggctagccaaactgacgctctacctgttggtttttgccctagcggctcgggttctccgcaatccccgtctgcgatcgctgctgttctcggtcgtcgtgatcacatcgctttttgtcagtgtctacggcctcaaccaatggatctacggcgttgaagagctggcgacttgggtggatcgcaactcggttgccgacttcacctcacgggtttacagctatctgggcaaccccaacctgctggctgcttatctggtgccgacgactgccttttctgcagcagcgatcggggtgtggcgcggctggctccccaagctgctggcgatcgctgcgacaggtgcgagcagcttatgtctgatcctcacctacagtcgcggtggctggctgggttttgtcgccatgatttttgtctgggcgttattagggctctactggtttcaaccccgtctacccgcaccctggcgacgctggctattcccagtcgtattgggtggactagtcgcggtgctcttggtggcggtgcttggacttgagccgttgcgcgtgcgcgtgttgagcatctttgtggggcgtgaagacagcagcaacaacttccggatcaatgtctggctggcggtgctgcagatgattcaagatcggccttggctgggcatcggccccggcaataccgcctttaacctggtttatcccctctatcaacaggcgcgctttacggcgttgagcgcctactccgtcccgctggaagtcgcggttgagggcggactactgggcttgacggccttcgcttggctgctgctggtcacggcggtgacggcggtgcggcaggtgagccgactgcggcgcgatcgcaatccccaagccttttggttgatggctagcttggccggtttggcaggaatgctgggtcacggtctgtttgataccgtgctctatcgaccggaagccagtacgctctggtggctctgtattggagcgatcgcgagtttctggcagccccaaccttccaagcaactccctccagaagccgagcattcagacgaaaaaatgtag

SEQ ID NO: 3 - protein, 467 aa, Synechococcus sp.
MTVWQTLTFAHYQPQQWGHSSFLHRLFGSLRAWRASSQLLVWSEALGGFLLAVVYGSAPFVPSSALGLGLAAIAAYWALLSLTDIDLRQATPIHWLVLLYWGVDALATGLSPVRAAALVGLAKLTLYLLVFALAARVLRNPRLRSLLFSVVVITSLFVSVYGLNQWIYGVEELATWVDRNSVADFTSRVYSYLGNPNLLAAYLVPTTAFSAAAIGVWRGWLPKLLAIAATGASSLCLILTYSRGGWLGFVAMIFVWALLGLYWFQPRLPAPWRRWLFPVVLGGLVAVLLVAVLGLEPLRVRVLSIFVGREDSSNNFRINVWLAVLQMIQDRPWLGIGPGNTAFNLVYPLYQQARFTALSAYSVPLEVAVEGGLLGLTAFAWLLLVTAVTAVRQVSRLRRDRNPQAFWLMASLAGLAGMLGHGLFDTVLYRPEASTLWWLCIGAIASFWQPQPSKQLPPEAEHSDEKM

SEQ ID NO: 4 - DNA, 1425 bp, Synechocystis sp.
atggtgtctcccatctctatctggcgatcgctgatgtttggcggtttttccccccaggaatggggccggggcagtgtgctccatcgtttggtgggctggggacagagttggatacaggctagtgtgctctggccccacttcgaggcattgggtacggctctagtggcaataatttttattgcggctcccttcacctccaccaccatgttgggcatttttatgctgctctgtggagccttttgggctctgctgacctttgctgatcaaccagggaagggtttgactcccatccatgttttagtttttgcctactggtgcatttcggcgatcgccgtgggattttctccggtaaaaatggcggcggcgtcggggttagcgaaattaacagctaatttatgtctgtttctactggcggcgaggttattgcaaaacaaacaatggttgaaccggttagtaaccgttgttttactggtagggctattggtggggagttacggtctgcgacaacaggtggacggggtagaacagttagccacttggaatgaccccacctctaccttggcccaggccactagggtatatagctttttaggtaatcccaatctcttggcggcttacctggtgcccatgacgggtttgagcttgagtgccctggtggtatggcgacggtggtggcccaaactgctgggagcaaccatggtgattgttaacctactctgtctcttttttacccagagccggggcggttggctagcagtgctggccctgggagctaccttcctggccctttgttacttctggtggttaccccaattacccaaattttggcaacggtggtctttgcccctggcgatcgccgtggcggttatattaggtgggggagcgttgattgcggtggaaccgattcgactcagggccatgagcatttttgctgggcgggaagacagcagtaataatttccgcatcaatgtttgggaaggggtaaaagccatgatccgagcccgccctatcattggcattggcccaggtaacgaagcctttaaccaaatttatccttactatatgcggccccgcttcaccgccctgagtgcctattccatttacctagaaattttggtggaaacgggtgtagttggttttacctgtatgctctggctgttggccgttaccctaggcaaaggcgtagaactggttaaacgctgtcgccaaaccctcgccccggaaggcatctggattatgggggctttagcggcgatcatcggtttgttggtccacggcatggtagatacagtctggtaccgtcccccggtgagcactttgtggtggttgctagtggccattgttgctagtcagtgggccagcgcccaggcccgtttggaggccagtaaagaagaaaatgaggacaaacctcttcttgcttcataa

SEQ ID NO: 5 - protein, 474 aa, Synechocystis sp.
MVSPISIWRSLMFGGFSPQEWGRGSVLHRLVGWGQSWIQASVLWPHFEALGTALVAIIFIAAPFTSTTMLGIFMLLCGAFWALLTFADQPGKGLTPIHVLVFAYWCISAIAVGFSPVKMAAASGLAKLTANLCLFLLAARLLQNKQWLNRLVTVVLLVGLLVGSYGLRQQVDGVEQLATWNDPTSTLAQATRVYSFLGNPNLLAAYLVPMTGLSLSALVVWRRWWPKLLGATMVIVNLLCLFFTQSRGGWLAVLALGATFLALCYFWWLPQLPKFWQRWSLPLAIAVAVILGGGALIAVEPIRLRAMSIFAGREDSSNNFRINVWEGVKAMIRARPIIGIGPGNEAFNQIYPYYMRPRFTALSAYSIYLEILVETGVVGFTCMLWLLAVTLGKGVELVKRCRQTLAPEGIWIMGALAAIIGLLVHGMVDTVWYRPPVSTLWWLLVAIVASQWASAQARLEASKEENEDKPLLAS

SEQ ID NO: 6 - protein, 475 aa, Anabaena PCC7120
MNLVWQRFTLSSLPLKQFLATSYLHRFLVGLLSSWRQTSFLLQWGDMIAAALLSLIYVLAPFVSSTLVGVLLIACVGFWLLLTLSDEPSSNNNSLVTPIHLLVLLYWGIAAVATALSPVKKAALTDLLTLTLYLLLFALCARVLRSPRLRSWIITLYLSASLVVSIYGMRQWRFGAPPLATWVDPESTLSKTTRVYSYLGNPNLLAGYLVPAVIFSLMAVFVWQGWARKSLAVTMLFVNTACLIFTYSRGGWIGLVVAVLGATALLVDWWSVQMPPFWRTWSLPILLGGLIGVLLIAVLFVEPVRFRVLSIFADRQDSSNNFRRNVWDAVFEMIRDRPIIGIGPGHNSFNKVYPLYQRPRYSALSAYSIFLEVAVEMGFVGLACFLWLIIVTINTAFVQLRQLRQSANVQGFWLVGALATLLGMLAHGTVDTIWFRPEVNTLWWLMVALIASYWTPLSANQCQELNLFKEEPTSN

SEQ ID NO: 7 - protein, 472 aa, Nostoc punctiforme
MNLVWQLFTLSSLPLKEYLATSYVHRSLVGLLSSWRQTSVLIQWGDAIAAVLLSSIYALAPFASSTLVGLLLVACVGFWLLLTLSDEVTPANVSSVTPIHLLVLLYWGIAVIATALSPVKKAALNDLGTLTLYLLLFALCARVLRSPRLRSWILTLYLHVSLIVSVYGLRQWFFGATALATWVDPESPLSKTTRVYSYLGNPNLLAGYLLPAVIFSLVAIFAWQSWLKKALALTMLIVNTACLILTFSRGGWIGLVVAVLAVMALLVFWKSVEMPPFWRTWSLPIVLGGLIGILLLAVIFVEPVRLRVFSIFADRQDSSNNFRRNVWDAVFEMIRDRPIFGIGPGHNSFNKVYPLYQHPRYTALSAYSILFEVTVETGFVGLACFLWLIIVTFNTALLQVRRLRRLRSVEGFWLIGAIAILLGMLAHGTVDTVWYRPEVNTLWWLIVALIASYWTPLTQNQTNPSNPEPAVN

SEQ ID NO: 8 - DNA, 1425 bp, Anabaena PCC7120
atgaatttagtctggcaacgatttactttatcttctttacctctaaaacagtttctagctacaagttacttacatcggttcctagtgggactgttatcttcttggcggcaaactagtttcttacttcagtggggagacatgattgcagctgcgttactcagcttgatatatgttttggctccctttgtctctagtactctcgttggtgtgctgctgatagcttgtgtaggtttttggttattgttgactttatctgatgaaccttcatcaaacaataactcccttgttactcccatacacctgttggtgttgctctattggggaattgctgctgtagcaacggcattatcaccagtcaagaaggcagcattaactgatttgttaaccttgactttgtatttgctactatttgctctttgtgccagggtgctgagatcgccgcgtctgaggtcttggatcattaccctctacctatctgcatcactggttgtcagtatatatggaatgcgacaatggcgttttggtgcgcccccactggcgacttgggttgatccagagtccaccttgtctaaaaccacaagggtttacagttatttaggcaatcccaatttgttggctggttatttagtaccggcggtgatttttagcctcatggcagtttttgtctggcagggctgggcaagaaaatctttagctgtaacaatgctgtttgtaaacactgcttgcctaatttttacttatagtcgtggcggctggattggtcttgtggtagcagtcttaggggcgacggcattgctagttgattggtggagtgtgcaaatgccgcctttttggcgaacctggtcattacccatacttttgggcggtttgatcggggtattgttgattgcggtgttatttgtcgagccagtccggtttcgagttctcagtatttttgccgatcgccaagatagcagcaataattttcgccgcaacgtgtgggatgctgtttttgagatgatccgcgatcgcccaattattggtattggccctggtcataattcttttaataaagtctaccctctttaccaaagacctcgttatagtgctttaagtgcctattccatcttcctagaggtggctgtagaaatgggttttgttggactagcttgctttctctggttaattatcgtcactattaatacagcattcgttcagctacgccaactgcgccaatctgccaatgtgcaaggattttggttggtgggtgccttagccacattgctgggaatgctggctcacggtacggtagacactatatggtttcgtccggaagttaatactctttggtggttaatggttgctctcattgctagctattggacacctttatccgcaaaccaatgtcaagaactcaatttatttaaggaagaacccacaagcaac

SEQ ID NO: 9 - DNA, 1419 bp, Nostoc punctiforme
atgaatttagtctggcaactatttactttatcatctttaccgctcaaagaatatcttgctaccagttacgtacaccgttctctggtgggactgttaagctcttggcggcaaaccagcgtcttgattcagtggggagatgcgatagcagctgtattactcagctcaatatatgcccttgcaccttttgcttcgagtactttggtaggtttattgctggtcgcttgtgtgggattttggctattgttgactttatctgatgaagtcacaccagcaaatgtctcgtcagtcactcccattcatctactggtattgctctactggggaattgccgtaatcgcaacagcattatcaccagtgaaaaaagcggcacttaacgacttgggaactttgaccttgtatttgctactatttgccctttgtgccagggtattaaggtcgcctcgcctccggtcttggattctcaccctttatctgcacgtatcgttaattgtcagtgtctatggattgcggcaatggttttttggagccacagcactggcaacttgggttgatccggaatctcctctgtctaagactacaagagtctacagttatttaggaaatcccaacttattggctggatacctcttaccagcagtaatttttagcttggtggcaatttttgcatggcaaagttggctcaaaaaagccttagcattaacaatgttgattgtcaatactgcctgcctgatcctgacttttagtcgtggcggttggattggactagtggtggcagttttggcggtgatggcattgctagttttttggaagagtgtggaaatgcctcctttttggcgtacttggtcgctgcccattgtcttaggaggtttaattgggatattactgttagcagtgatatttgtagagccagttcgcctgcgggtgttcagcatttttgctgaccgtcaagatagtagtaataattttcgtcgaaatgtgtgggatgctgtctttgagatgattcgcgatcgcccaattttcggtattggccctggtcacaactcttttaataaagtttatccgctctaccaacaccctcggtacactgctttaagtgcttattcgattttgtttgaagtgactgtagaaactgggtttgttggtttagcttgctttctctggctaataatcgtcacatttaatacggcgcttttgcaagtacgacgattgcgacgattgagaagtgtagagggattttggttaattggagcgatcgctattttgttgggtatgctcgctcacggcactgtagatactgtctggtatcgtcctgaagtcaataccctctggtggctcatcgttgctttaattgccagctactggacacctttaactcaaaaccagacaaatccatctaacccagaaccagcagtaaactaa

(19) United States
(12) Patent Application Publication
(10) Pub. No.: US 2003/0037356 A1
(43) Pub. Date: Feb. 20, 2003

(54) Plants characterized by enhanced growth and methods and nucleic acid constructs useful for generating same

(75) Inventors: Aaron Kaplan, Jerusalem (IL); Judy Lieman-Hurwitz, Jerusalem (IL); Shimon Rachmilevitch, Jerusalem (IL); Daniella Schatz, Jerusalem (IL); Ron Mittler, Jerusalem (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Yissum Research Development Company Of The Hebrew University Of Jerusalem

(21) Appl. No.: 09/828,173

(22) Filed: Apr. 9, 2001

(51) Int. Cl. C12N 15/82; C12N 15/31; C12P 21/00

(52) U.S. Cl. 800/278; 536/23.4; 435/69.8; 435/419; 800/288; 800/290; 536/23.7

(57) ABSTRACT

A method of enhancing growth and/or commercial yield of a plant is provided. The method is effected by expressing within the plant a polypeptide including an amino acid sequence at least 60% homologous to that set forth in SEQ ID NOs:3, 5, 6 or 7.

### FIELD AND BACKGROUND OF THE INVENTION

The present invention relates to plants characterized by enhanced growth and to methods and nucleic acid constructs useful for generating same.

Growth and productivity of crop plants are the main parameters of concern to a commercial grower. Such parameters are affected by numerous factors including the nature of the specific plant and allocation of resources within it, availability of resources in the growth environment and interactions with other organisms including pathogens.

Growth and productivity of most crop plants are limited by the availability of CO₂ to the carboxylating enzyme ribulose 1,5-bisphosphate carboxylase/oxygenase (Rubisco). Such availability is determined by the ambient concentration of CO₂ and stomatal conductance, and the rate of CO₂ fixation by Rubisco as determined by the Km(CO₂) and Vmax of this enzyme [31-33].

In C3 plants, the concentration of CO₂ at the site of Rubisco is lower than the Km(CO₂) of the enzyme, particularly under water stress conditions. As such, these crop plants exhibit a substantial decrease in growth and productivity when exposed to low CO₂ conditions induced by, for example, stomatal closure which can be caused by water stress.

Many photosynthetic microorganisms are capable of concentrating CO₂ at the site of Rubisco to thereby overcome the limitation imposed by the low affinity of Rubisco for CO₂ [34].

Higher plants of the C4 and the CAM physiological groups can also raise the concentration of CO₂ at the site of Rubisco by means of dual carboxylations which are spatially (in C4) or timely (in CAM) separated.

Since plant growth and productivity especially in C3 crop plants are highly dependent on CO₂ availability to Rubisco and fixation rates, numerous attempts have been made to genetically modify plants in order to enhance CO₂ concentration or fixation therein in hopes that such modification would lead to an increase in growth or yield.

As such, numerous studies attempted to introduce the CO₂ concentrating mechanisms of photosynthetic bacteria or C4 plants into C3 plants, so far with little or no success.

For example, studies attempting to genetically modify Rubisco in order to raise its affinity for CO₂ [35] and transformation of a C3 plant (rice) with several genes responsible for C4 metabolism have been described [36-40].

Although theoretically such approaches can lead to enhanced CO₂ fixation in C3 plants, results obtained from such studies have been disappointing.

There is thus a widely recognized need for, and it would be highly advantageous to have, a method of generating plants exhibiting enhanced growth and/or increased commercial yields.

### SUMMARY OF THE INVENTION

According to one aspect of the present invention there is provided method of enhancing growth and/or commercial yield of a plant, the method comprising expressing within the plant a polypeptide including an amino acid sequence at least 60% homologous to that set forth in SEQ ID NOs:3, 5, 6 or 7.

According to another aspect of the present invention there is provided a transformed plant expressing a polypeptide including an amino acid sequence at least 60% homologous to that set forth in SEQ ID NOs:3, 5, 6 or 7 the transformed plant characterized by an enhanced growth as compared to similar non transformed plant grown under similar growth conditions.

According to further features in preferred embodiments of the invention described below, the plant is grown in an environment characterized by humidity lower than 40%.

According to still further features in the described preferred embodiments the plant is grown in an environment characterized by a CO₂ concentration similar to or lower than in air, (approximately 0.035% CO₂ in air, and 10 micromolar CO₂ in solution).

According to still further features in the described preferred embodiments expressing the polypeptide within the plant is effected by transforming at least a portion of the plant cells with a nucleic acid construct including a polynucleotide region encoding the polypeptide.

According to still further features in the described preferred embodiments transforming is effected by a method selected from the group consisting of Agrobacterium mediated transformation, viral infection, electroporation and particle bombardment.

According to still further features in the described preferred embodiments the amino acid sequence is as set forth by SEQ ID NOs:3, 5, 6, 7.

According to still further features in the described preferred embodiments the nucleic acid construct further includes a second polynucleotide region encoding a transit peptide.

According to still further features in the described preferred embodiments the nucleic acid construct further includes a promoter sequence for directing transcription of the first polynucleotide region.

According to still further features in the described preferred embodiments the nucleic acid construct further includes a promoter sequence for directing transcription of the first and the second polynucleotide regions.

According to still further features in the described preferred embodiments the promoter is functional in eukaryotic cells.

According to still further features in the described preferred embodiments the promoter is selected from the group consisting of a constitutive promoter, an inducible promoter, a developmentally regulated promoter and a tissue specific promoter.

According to still further features in the described preferred embodiments the plant is a C3 plant.

According to still further features in the described preferred embodiments the C3 plant is selected from the group consisting of tomato, soybean, potato, cucumber, cotton, wheat, rice, barley, sunflower, banana, tobacco, lettuce, cabbage, petunia, solidago and poplar. According to still further features in the described preferred embodiments the plant is a C4 plant.

According to still further features in the described preferred embodiments the C4 plant is selected from the group consisting of corn, sugar cane and sohrgum.

According to still further features in the described preferred embodiments the plant expressing the polypeptide is characterized by a growth rate which is at least 10% higher than that of a similar plant not expressing the polypeptide when both are grown under similar growth conditions where CO₂ becomes limiting.

According to still further features in the described preferred embodiments the growth rate is determined by at least one growth parameter selected from the group consisting of increased fresh weight, increased dry weight, increased root growth, increased shoot growth and flower development over time.

According to still further features in the described preferred embodiments the transformed plant is further characterized by an increased commercial yield as compared to similar non transformed plant grown under similar CO₂ limiting conditions.

According to yet another aspect of the present invention there is provided a nucleic acid expression construct comprising: (a) a first polynucleotide region encoding a polypeptide including an amino acid sequence at least 60% homologous to that set forth by SEQ ID NOs:3, 5, 6 or 7; and (b) a second polynucleotide region functional as a promoter and being for directing the transcription of the first polynucleotide region in eukaryotic cells.

According to still further features in the described preferred embodiments the promoter is selected from the group consisting of a constitutive promoter, an inducible promoter and a tissue specific promoter.

According to still further features in the described preferred embodiments the promoter is a plant promoter.

According to still further features in the described preferred embodiments the first polynucleotide region further encodes a transit peptide being translationally fused to the polypeptide.

The present invention successfully addresses the shortcomings of the presently known configurations by providing plants characterized by enhanced growth and to methods and nucleic acid constructs useful for generating same.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic representation of a genomic region in Synechococcus sp. PCC 7942 where an insertion (indicated by a star) of an inactivation library fragment led to the formation of mutant L-2. DNA sequence is available in the GenBank, Accession number U62616. Restriction sites are marked as: A-ApaI, B-BamHI, Ei-EcoRI, E-EcoRV, H-HincII, Hi-HindIII, K-KpnI, M-MfeI, N-NheI, T-TaqI. Underlined letters represent the terminate position of the DNA fragments that were used as probes. Relevant fragments isolated from an EMBL3 library are marked E1, E2 and E3. P1 and P2 are fragments obtained by PCR. Triangles indicate sites where a cartridge encoding Kan^{r} was inserted. Open reading frames are marked by an arrow and their similarities to other proteins are noted. Sll and slr (followed by four digits) are the homologous genes in Synechocystis sp. PCC 6803 [23]; YZ02-myctu, Accession No. Q10536; ICC, Accession No. P36650; Y128-SYNP6, Accession No. P05677; YGGH Accession No. P44648; Ribosome binding factor A homologous to sll0754 and to P45141; O-acetylhomoserine sulfhydrylase homologous to sll0077 and NifS. ORF280 started upstream of the schematic representation presented herein.

FIG. 2 shows nucleic acid sequence alignment between ORF467 (ICTB, SEQ ID NO:2) and slrl155 (SLR, SEQ ID NO:4). Vertical lines indicate nucleotide identity. Gaps are indicated by hyphens. Alignment was performed using the Blast software where gap penalty equals 10 for existence and 10 for extension, average match equals 10 and average mismatch equals −5. Identical nucleotides equals 56%.

FIG. 3 shows amino acid sequence alignment between the IctB protein (ICTB, SEQ ID NO:3) and the protein encoded by slr1515 (SLR, SEQ ID NO:5). Identical amino acids are marked by their single letter code between the aligned sequences, similar amino acids are indicated by a plus sign. Alignment was performed using the Blast software where gap open penalty equals 11, gap extension penalty equals 1 and matrix is blosum62. Identical amino acids equals 47%, similar amino acids equals 16%, total homology equals 63%.

FIGS. 4a-b are graphs showing the rates of CO₂ and of HCO₃⁻ uptake by Synechococcus PCC 7942 (4a) and mutant IL-2 (4b) as a function of external Ci concentration. LC and HC are cells grown under low (air) or high CO₂ (5%CO₂ in air), respectively. The rates were assessed from measurements during steady state photosynthesis using a membrane inlet mass spectrometer (MIMS) [6, 7, 22].

FIG. 5 presents DNA sequence homology comparison of a region of ictB found in Synechococcus PCC 7942 and in mutant IL-2. This region was duplicated in the mutant due to a single cross-over event. Compared with the wild type, one additional nucleotide and a deletion of six nucleotides were found in the BamHI side, and 4 nucleotides were deleted in the ApaI side (see FIG. 1). These changes resulted in stop codons in IctB after 168 or 80 amino acids in the BamHI and ApaI sides, respectively. The sequence shown by this Figure starts from the 69^{th} amino acid of ictB.

FIG. 6 illustrates the ictB construct used in generating the transgenic plants of the present invention, including a 35S promoter, the transit peptide (TP) from the small subunit of pea Rubisco (nucleotide coordinates 329-498 of GeneBank Accession number x04334 where we replaced the G in position 498 with a T, the ictB coding region, the NOS termination and kanamycin-resistance (Kn^{R}) within the binary vector pBI121 from Clontech.

FIG. 7 is a Northern blot analysis of transgenic and wild type (w) Arabidopsis and tobacco plants using both ictB and 18S rDNA as probes.

FIG. 8 illustrates the rate of photosynthesis as affected by the intercellular concentration of CO₂ in wild type and the transgenic tobacco plants of the present invention; plants 1 and 11 are transgenic.

FIG. 9 illustrates growth experiments conducted on both transgenic (A, B and C) and wild type (WT) Arabidopsis plants. Each growth pot included one wild type and three transgenic plants. Data are provided as the average dry weight of the plants +/− S.D. Growth conditions are described in the Examples section.

### DESCRIPTION OF THE PREFERRED EMBODIMENTS

The present invention is of a method of generating plants characterized by enhanced growth and/or fruit yield and/or flowering rate, of plants generated thereby and of nucleic acid constructs utilized by such a method. Specifically, the present invention can be used to substantially increase the growth rate and/or fruit yield of C3 plants especially when grown under conditions characterized by low humidity and/or a low CO₂ concentration.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Increasing the growth size/rate and/or commercial yield of crop plants is of paramount importance especially in regions in which growth/cultivation conditions are suboptimal due to a lack of, for example, water.

While reducing the present invention to practice the inventors have discovered that plants expressing exogenous polynucleotides encoding a putative cyanobacterial inorganic carbon transporter are characterized by enhanced growth, especially when grown under conditions characterized by low humidity or low CO₂ concentrations.

Thus, according to the present invention there is provided a transformed plant expressing a polypeptide including an amino acid sequence which is at least 60% homologous to that set forth in SEQ ID NO:3, 5, 6 or 7.

The transformed plant of the present invention is characterized by enhanced growth as compared to similar non transformed plant grown under similar growth conditions.

As used herein, the phrase “enhanced growth” refers to an enhanced growth rate, or to an increased growth size/weight of the whole plant or preferably the commercial portion of the plant (increased yield) as determined by fresh weight, dry weight or size of the plant or commercial portion thereof.

As is further detailed in the Examples section which follows, the transformed plants of the present invention exhibit, for example, a growth rate which is at least 10% higher than that of a similar non transformed plant when both plants are grown under similar growth conditions.

According to a preferred embodiment of the present invention, the polypeptide is at least 60%, preferably at least 65%, more preferably at least 70%, still more preferably at least 75%, yet more preferably at least 80%, more preferably at least 85%, more preferably at least 90%, yet more preferably at least 95%, ideally 95-100% homologous (identical+similar) to SEQ ID NO:3, 5, 6 or 7 or a portion thereof as determined using the Blast software where gap open penalty equals 11, gap extension penalty equals 1 and matrix is blosum62.

According to preferred embodiments of the present invention, the growth conditions are characterized by humidity of less than 40% and/or CO₂ concentration which is lower than in air.

The transformed plant of the present invention can be any plant including, but not limited to, C3 plants such as, for example, tomato, soybean, potato, cucumber, cotton, wheat, rice, barley or C4 plants, such as, for example, corn, sugar cane, sohrgum and others.

The transformed plant of the present invention is generated by introducing a nucleic acid molecule or polynucleotide encoding the polypeptide(s) described above into cells of the plant.

Such a nucleic acid molecule or polynucleotide can have a sequence corresponding to at least a portion of SEQ ID NO:2, 4, 8 or 9 the portion encoding a polypeptide contributing the increased growth trait.

Alternatively or additionally the nucleic acid molecule can have a sequence which is at least 60%, preferably at least 65%, more preferably at least 70%, still more preferably at least 75%, yet more preferably at least 80%, more preferably at least 85%, more preferably at least 90%, yet more preferably at least 95%, ideally 95-100% identical to that portion, as determined using the Blast software where gap penalty equals 10 for existence and 10 for extension, average match equals 10 and average mismatch equals −5. It will be appreciated in this respect that SEQ ID NO:2, 4, 8 or 9 can be readily used to isolate homologous sequences which can be tested as described in the Examples section that follows for their bicarbonate transport activity. Methods for isolating such homologous sequences are extensively described in, for example, Sambrook et al. [9] and may include hybridization and PCR amplification.

Still alternatively or additionally the nucleic acid molecule can have a sequence capable of hybridizing with the portion of SEQ ID NO:2, 4, 8 or 9. Hybridization for long nucleic acids (e.g., above 200 bp in length) is effected according to preferred embodiments of the present invention by stringent or moderate hybridization, wherein stringent hybridization is effected by a hybridization solution containing 10% dextrane sulfate, 1 M NaCl, 1% SDS and 5×10⁶ cpm ³²p labeled probe, at 65° C., with a final wash solution of 0.2×SSC and 0.1% SDS and final wash at 65° C.; whereas moderate hybridization is effected by a hybridization solution containing 10% dextrane sulfate, 1 M NaCl, 1% SDS and 5×10⁶ cpm ³²p labeled probe, at 65° C., with a final wash solution of 1×SSC and 0.1% SDS and final wash at 50° C.

Preferably, the polypeptide encoded by the nucleic acid molecule of the present invention includes an N terminal transit peptide fused thereto which serves for directing the polypeptide to a specific membrane. Such a membrane can be, for example, the cell membrane, wherein the polypeptide will serve to transport bicarbonate from the apoplast into the cytoplasm, or, such a membrane can be the outer and preferably the inner chloroplast membrane. Transit peptides which function as herein described are well known in the art. Further description of such transit peptides is found in, for example, Johnson et al. The Plant Cell (1990) 2:525-532; Sauer et al. EMBO J. (1990) 9:3045-3050; Mueckler et al. Science (1985) 229:941-945; Von Heijne, Eur. J. Biochem. (1983) 133:17-21; Yon Heijne, J. Mol. Biol. (1986) 189:239-242; Iturriaga et al. The Plant Cell (1989) 1:381-390; McKnight et al., Nucl. Acid Res. (1990) 18:4939-4943; Matsuoka and Nakamura, Proc. Natl. Acad. Sci. USA (1991) 88:834-838. A recent text book entitled “Recombinant proteins from plants”, Eds. C. Cunningham and A. J. R. Porter, 1998 Humana Press Totowa, N.J. describe methods for the production of recombinant proteins in plants and methods for targeting the proteins to different compartments in the plant cell. The book by Cunningham and Porter is incorporated herein by reference. It will however be appreciated by one of skills in the art that a large number of membrane integrated proteins fail to poses a removable transit peptide. It is accepted that in such cases a certain amino acid sequence in said proteins serves not only as a structural portion of the protein, but also as a transit peptide.

Preferably, the nucleic acid molecule of the present invention is included within a nucleic acid construct designed as a vector for transforming plant cells thereby enabling expression of the nucleic acid molecule within such cells.

Plant expression can be effected by introducing the nucleic acid molecule of the present invention (preferably using the nucleic acid construct) downstream of a plant promoter present in endogenous genomic or organelle polynucleotide sequences (e.g., chloroplast or mitochondria), thereby enabling expression thereof within the plant cells.

In such cases, the nucleic acid construct further includes sequences which enable to “knock-in” the nucleic acid molecule into specific or random polynucleotide regions of such genomic or organelle polynucleotide sequences.

Preferably, the nucleic acid construct of the present invention further includes a plant promoter which serves for directing expression of the nucleic acid molecule within plant cells.

As used herein in the specification and in the claims section that follows the phrase “plant promoter” includes a promoter which can direct gene expression in plant cells (including DNA containing organelles). Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric.

Thus, the plant promoter employed can be a constitutive promoter, a tissue specific promoter, an inducible promoter or a chimeric promoter.

Examples of constitutive plant promoters include, without limitation, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter.

Examples of tissue specific promoters include, without being limited to, bean phaseolin storage protein promoter, DLEC promoter, PHSβ promoter, zein storage protein promoter, conglutin gamma promoter from soybean, AT2S1 gene promoter, ACT11 actin promoter from Arabidopsis, napA promoter from Brassica napus and potato patatin gene promoter.

The inducible promoter is a promoter induced by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

The nucleic acid construct of the present invention preferably further includes additional polynucleotide regions which provide a broad host range prokaryote replication origin; a prokaryote selectable marker; and, for Agrobacterium transformations, T DNA sequences for Agrobacterium-mediated transfer to plant chromosomes. Where the heterologous sequence is not readily amenable to detection, the construct will preferably also have a selectable marker gene suitable for determining if a plant cell has been transformed. A general review of suitable markers for the members of the grass family is found in Wilmink and Dons, Plant Mol. Biol. Reptr. (1993) 11:165-185.

Suitable prokaryote selectable markers include resistance toward antibiotics such as ampicillin, kanamycin or tetracycline. Other DNA sequences encoding additional functions may also be present in the vector, as is known in the art.

Sequences suitable for permitting integration of the heterologous sequence into the plant genome are also recommended. These might include transposon sequences as well as Ti sequences which permit random insertion of a heterologous expression cassette into a plant genome.

The nucleic acid construct of the present invention can be utilized to stably or transiently transform plant cells. In stable transformation, the nucleic acid molecule of the present invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto el al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, Tm and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, N.Y., pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Thus, the present invention provides methods, nucleic acid constructs and transformed plants generated using such methods and constructs, which transformed plants are characterized by an enhanced growth rate and/or increased commercial yield.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

### ictB isolation and characterization Materials and Experimental Methods

Growth Conditions:

Cultures of Synechococcus sp. strain PCC 7942 and mutant IL-2 thereof were grown at 30° C. in BG₁₁ medium supplemented with 20 mM Hepes-NaOH pH 7.8 and 25 μg mL⁻¹ kanamycin (in the case of the mutant). The medium was aerated with either 5% v/v CO₂ in air (high CO₂) or 0.0175% v/v CO₂ in air (low CO₂) which was prepared by mixing air with CO₂-free air at a 1:1 ratio. Escherichia coli (strain DH5α) were grown on an LB medium [9] supplemented with either kanamycin (50 μg/mL) or ampicillin (50 μg/mL) when required.

Measurements of Photosynthesis and Ci Uptake:

The rates of inorganic carbon (Ci)-dependent O₂ evolution were measured by an O₂ electrode as described elsewhere [10] and by a membrane inlet mass spectrometer (MIMS, [6, 11]). The MIMS was also used for assessments of CO₂ and HCO₃⁻ uptake during steady state photosynthesis [6]. Ci fluxes following supply of CO₂ or HCO₃⁻ were determined by the filtering centrifugation technique [10]. High-CO₂ grown cells in the log phase of growth were transferred to either low or high CO₂ 12 hours before conducting the experiments. Following harvest, the cells were resuspended in 25 mM Hepes-NaOH pH 8.0 and aerated with air (Ci concentration was about 0.4 mM) under light flux of 100 μmol photon quanta m⁻² s⁻¹. Aliquots were withdrawn, immediately placed in microfuge tubes and kept under similar light and temperature conditions. Small amounts of ¹⁴C—CO₂ or ¹⁴C—HCO₃⁻ which did not affect the final Ci concentration, were injected, and the Ci uptake terminated after 5 seconds by centrifugation.

General DNA Manipulations:

Genomic DNA was isolated as described elsewhere [12]. Standard recombinant DNA techniques were used for cloning and Southern analyses [12-13] using the Random Primed DNA Labeling Kit or the DIG system (Boehringer, Mannheim). Sequence analysis was performed using the Dye Terminator cycle sequencing kit, ABI Prism (377 DNA sequencing Perkin Elmer). The genomic library used herein was constructed using a Lambda EMBL3/BamHI vector kit available from Stratagene (La Jolla, Calif.).

Construction and Isolation of Mutant IL-2:

A modification of the method developed by Dolganov and Grossman [14] was used to raise and isolate new high-CO₂-requiring mutants [4, 5]. Briefly, genomic DNA was digested with TaqI and ligated into the Acc site of the polylinker of a modified Bluescript SK plasmid. The bluescript borne gene for conferring ampicillin resistance was inactivated by the insertion of a cartridge encoding kanamycin resistance (Kan^{r}, [8]) (within the ScaI site). Synechococcus sp. strain PCC 7942 cells were transfected with the library [12]. Single crossover events which conferred Kan^{r} led to inactivation of various genes. The Kan^{r} cells were exposed to low CO₂ conditions for 8 hours for adaptation, followed by an ampicillin treatment (400 μg/mL) for 12 hours. Cells capable of adapting to low CO₂ and thus able to grow under these conditions were eliminated by this treatment. The high-CO₂-requiring mutant, IL-2, unable to divide under low CO₂ conditions, survived, and was rescued following the removal of ampicillin and growth in the presence of high CO₂ concentration.

Cloning of the Relevant Impaired Genomic Region From Mutant IL-2:

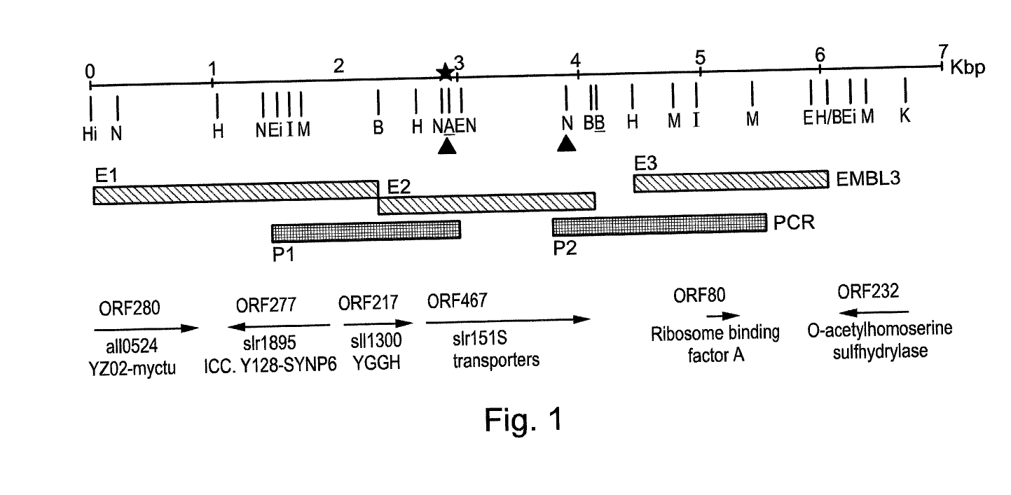

DNA isolated from the mutant was digested with ApaI located on one side of the AccI site in the polylinker; with BamHII or EcoRI, located on the other side of the AccI site; or with MfeI that does not cleave the vector or the Kan^{r} cartridge. These enzymes also cleaved the genomic DNA. The digested DNA was self-ligated followed by transfection of competent E. coli cells (strain DH5α). Kan^{r} colonies carrying the vector sequences bearing the origin of replication, the Kan^{r} cartridge and part of the inactivated gene were then isolated. This procedure was used to clone the flanking regions on both sides of the vector inserted into the mutant. A 1.3 Kbp ApaI and a 0.8 Kbp BamHI fragments isolated from the plasmids (one ApaI site and BamHI site originated from the vector's polylinker) were used as probes to identify the relevant clones in an EMBL3 genomic library of a wild type genome, and for Southern analyses. The location of these fragments in the wild type genome (SEQ ID NO:1) is schematically shown in FIG. 1. The ApaI fragment is between positions 1600 to 2899 (of SEQ ID NO:1), marked as T and A in FIG. 1; the BamHI fragment is between positions 4125 to 4957 (of SEQ ID NO:1) marked as B and T in FIG. 1. The 0.8 Kbp BamHI fragment hybridized with the 1.6 Kbp HincII fragment (marked E3 in FIG. 1). The 1.3 Kbp ApaI fragment hybridized with an EcoRI fragment of about 6 Kbp. Interestingly, this fragment could not be cloned from the genomic library into E. coli. Therefore, the BamHI site was used (position 2348, SEQ ID NO:1, FIG. 1) to split the EMBL3 clone into two clonable fragments of 4.0 and 1.8 Kbp (E1 and E2, respectively, E1 starts from a Sau3A site upstream of the HindIII site positioned at the beginning of FIG. 1). Confirmation that these three fragments were indeed located as shown in FIG. 1 was obtained by PCR using wild type DNA as template, leading to the synthesis of fragments P1 and P2 (FIG. 1). Sequence analyses enabled comparison of the relevant region in IL-2 with the corresponding sequence in the wild-type.

Physiological Analysis of the IL-2 Mutant:

The IL-2 mutant grew nearly the same as the wild type cells in the presence of high CO₂ concentration but was unable to grow under low CO₂. Analysis of the photosynthetic rate as a function of external Ci concentration revealed that the apparent photosynthetic affinity of the IL-2 mutant was 20 mM Ci, which is about 100 times higher than the concentration of Ci at the low CO₂ conditions. The curves relating to the photosynthetic rate as a function of Ci concentration, in IL-2, were similar to those obtained with other high-CO₂-requiring mutants of Synechococcus PCC 7942 [16, 17]. These data suggested that the inability of IL-2 to grow under low CO₂ is due to the poor photosynthetic performance of this mutant.

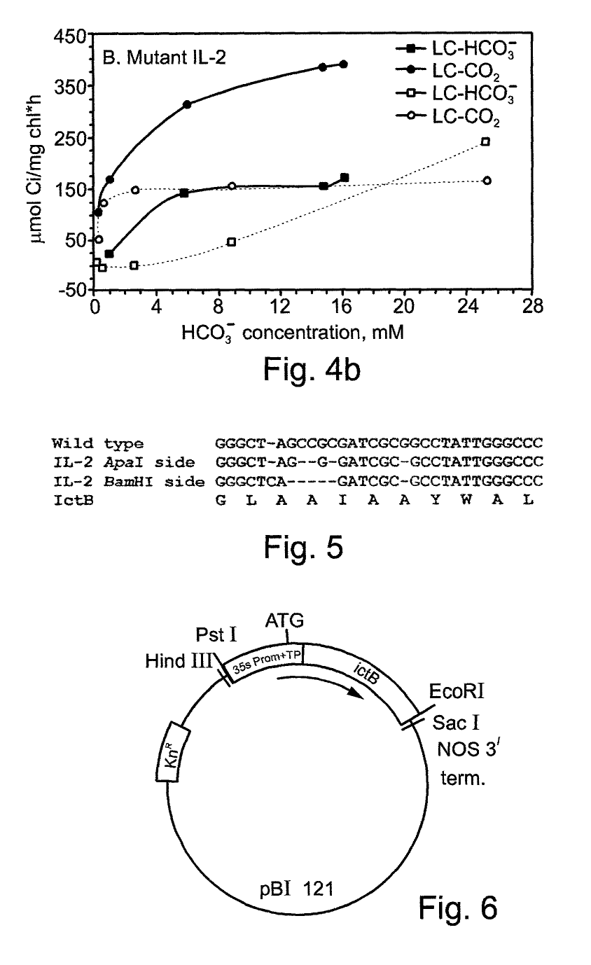

High-CO₂-requiring mutants showing such characteristics were recognized among mutants bearing aberrant carboxysomes [9, 10, 12, 18, 19] or defective in energization of Ci uptake [20, 21]. All the carboxysome-defective mutants characterized to date were able to accumulate Ci within the cells similarly to wild type cells. However, they were unable to utilize it efficiently in photosynthesis due to low activation state of rubisco in mutant cells exposed to low CO₂ [10]. This was not the case for mutant IL-2 which possessed normal carboxysomes but exhibited impaired HCO₃⁻ uptake (Table 1, FIGS. 4a-b). Measurements of ¹⁴Ci accumulation indicated that HCO₃⁻ and CO₂ uptake were similar in the high-CO₂-grown wild type and the mutant (Table 1).

TABLE 1

| | CO₂ Uptake | | HCO₃⁻ Uptake | |
|---|---|---|---|---|
| | High CO₂ | Low CO₂ | High CO₂ | Low CO₂ |
| WT | 31.6 | 53.9 | 30.9 | 182.0 |
| IL-2 | 26.6 | 39.2 | 32.2 | 61.1 |

The rate of CO₂ and of HCO₃⁻ uptake in Synechococcus sp. PCC 7942 and mutant IL-2 as affected by the concentration of CO₂ in the growth medium. The unidirectional CO₂ or HCO₃⁻ uptake of cells grown under high CO₂ conditions or exposed to low CO₂ for 12 hours is presented in μ mole Ci accumulated within the cells mg⁻¹ Chl h⁻¹. The results presented are the average of three different experiments, with four replicas in each experiment, the range of the data was within ±10% of the average. WT—wild type.

Uptake of HCO₃⁻ by wild type cells increased by approximately 6-fold following exposure to low CO₂ conditions for 12 hours. On the other hand, the same treatment resulted in only up to a 2-fold increase in HCO₃⁻ uptake for the L-2 mutant. Uptake of CO₂ increased by approximately 50% for both the wild type and the IL-2 mutant following transfer from high- to low CO₂ conditions. These data indicate that HCO₃₋ transport and not CO₂ uptake was impaired in mutant IL-2.

The Vₘₐₓ of HCO₃⁻ uptake, estimated by MIMS [7, 22] at steady state photosynthesis (FIG. 4a), were 220 and 290 μmol HCO₃⁻ mg⁻¹ Chl h⁻¹ for high- and low-CO₂-grown wild type, respectively, and the corresponding K_{½} (HCO₃⁻) were 0.3 and 0.04 mM HCO₃⁻, respectively. These estimates are in close agreement with those reported earlier [7]. In high-CO₂-grown mutant IL-2, on the other hand, the HCO₃⁻ transporting system was apparently inactive. The curve relating the rate of HCO₃⁻ transport as a function of its concentration did not resemble the expected saturable kinetics (observed for the wild type), but was closer to a linear dependence as expected in a diffusion mediated process (FIG. 4b). It was essential to raise the concentration of HCO₃⁻ in the medium to values as high as 25 mM in order to achieve rates of HCO₃⁻ uptake similar to the Vₘₐₓ depicted by the wild type.

The estimated Vₘₐₓ of CO₂ uptake by high-CO₂-grown wild type and IL-2 was similar for both at around 130-150 μmol CO₂ mg⁻¹ Chl h⁻¹ and the K_{½}(CO₂) values were around 5 μM (FIGS. 4a-b), indicating that CO₂ uptake was far less affected by the mutation in IL-2. Mutant cells that were exposed to low CO₂ for 12 hours showed saturable kinetics for HCO₃⁻ uptake suggesting the involvement of a carrier. However, the K_{½}(HCO₃⁻) was 4.5 mM HCO₃⁻(i.e., 15- and 100-fold lower than in high- and in low-CO₂-grown wild type, respectively) and the Vₘₐₓ was approximately 200 μmol HCO₃⁻ mg⁻¹ Chl h⁻¹. These data indicate the presence of a low affinity HCO₃⁻ transporter that is activated or utilized following inactivation of a high affinity HCO₃⁻ uptake in the mutant. The activity of the low affinity transporter resulted in the saturable transport kinetics observed in the low-CO₂-exposed mutant. These data further demonstrated that the mutant was able to respond to the low CO₂ signal.

The reason for the discrepancy between the data obtained by the two methods used, with respect to HCO₃⁻ uptake in wild type and mutant cells grown under high-CO₂-conditions, is not fully understood. It might be related to the fact that in the MIMS method HCO₃⁻ uptake is assessed as the difference between net photosynthesis and CO₂ uptake [6, 7, 22]. Therefore, at Ci concentrations below 3 mM, where the mutant did not exhibit net photosynthesis, HCO₃⁻ uptake was calculated as zero (FIGS. 4a-b). On the other hand, the filtering centrifugation technique, as used herein, measured the unidirectional HCO₃⁻ transport close to steady state via isotope exchange, which can explain some of the variations in the results. Not withstanding, the data obtained by both methods clearly indicates severe inhibition of HCO₃⁻ uptake in mutant cells exposed to low CO₂. It is interesting to note that while the characteristics of HCO₃⁻ uptake changed during acclimation of the mutant to low CO₂, CO₂ transport was not affected (FIGS. 4a-b). It is thus concluded that the high-CO₂-requiring phenotype of IL-2 is generated by the mutation of a HCO₃⁻ transporter rather than in non-acclimation to low CO₂.

Genomic Analysis of the IL-2 Mutant:

Since IL-2 is impaired in HCO₃⁻ transport, it was used to identify and clone the relevant genomic region involved in the high affinity HCO₃⁻ uptake. FIG. 1 presents a schematic map of the genomic region in Synechococcus sp. PCC 7942 where the insertion of the inactivating vector by a single cross over recombination event (indicated by a star) generated the IL-2 mutant. Sequence analysis (GenBank, accession No. U62616, SEQ ID NO:1) identified several open reading frames (identified in the legend of FIG. 1), some are similar to those identified in Synechocystis PCC 6803 [23]. Comparison of the DNA sequence in the wild type with those in the two repeated regions (due to the single cross over) in mutant IL-2, identified several alterations in the latter. This included a deletion of 4 nucleotides in the ApaI side and a deletion of 6 nucleotides but the addition of one bp in the BamHI side (FIG. 5). The reason(s) for these alterations is not known, but they occurred during the single cross recombination between the genomic DNA and the supercoiled plasmid bearing the insert in the inactivation library. The high-CO₂-requiring phenotype of mutant JR12 of Synechococcus sp. PCC 7942 also resulted from deletions of part of the vector and of a genomic region, during a single cross over event, leading to a deficiency in purine biosynthesis under low CO₂[24].

The alterations depicted in FIG. 5 resulted in frame shifts which led to inactivation of both copies of ORF467 (nucleotides 2670-4073 of SEQ ID NO:1, SEQ ID NO:2) in IL-2. Insertion of a Kan^{r} cartridge within the EcoRV or NheI sites in ORF467, positions 2919 and 3897 (SEQ ID NO:1), respectively (indicated by the triangles in FIG. 1), resulted in mutants capable of growing in the presence of kanamycin under low CO₂ conditions, though significantly (about 50%) slower than the wild type. Southern analyses of these mutants clearly indicated that they were merodiploids, i.e., contained both the wild type and the mutated genomic regions.

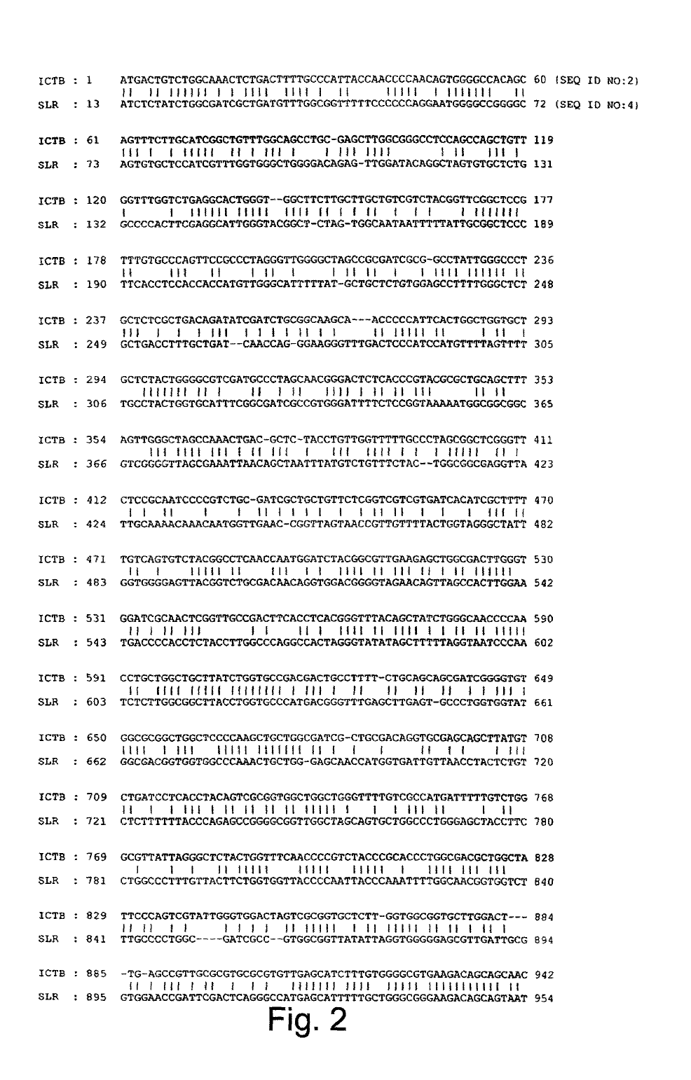

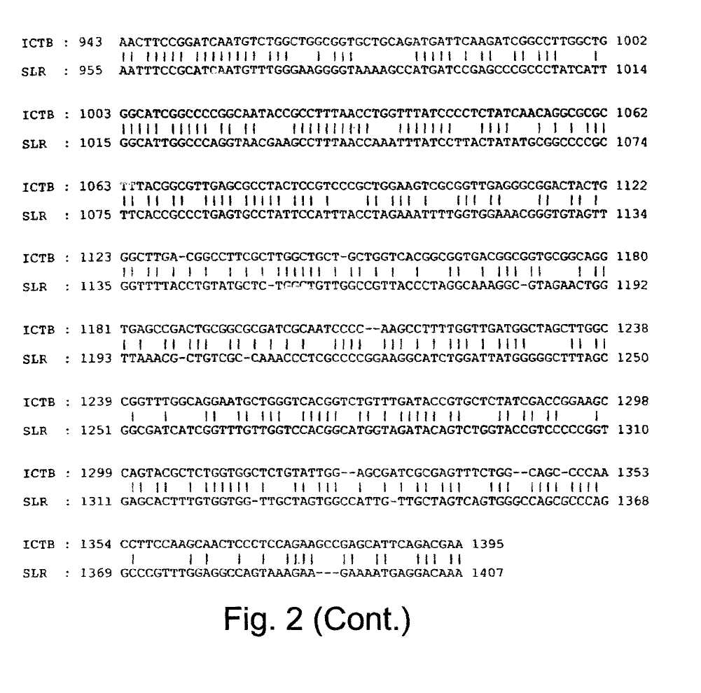

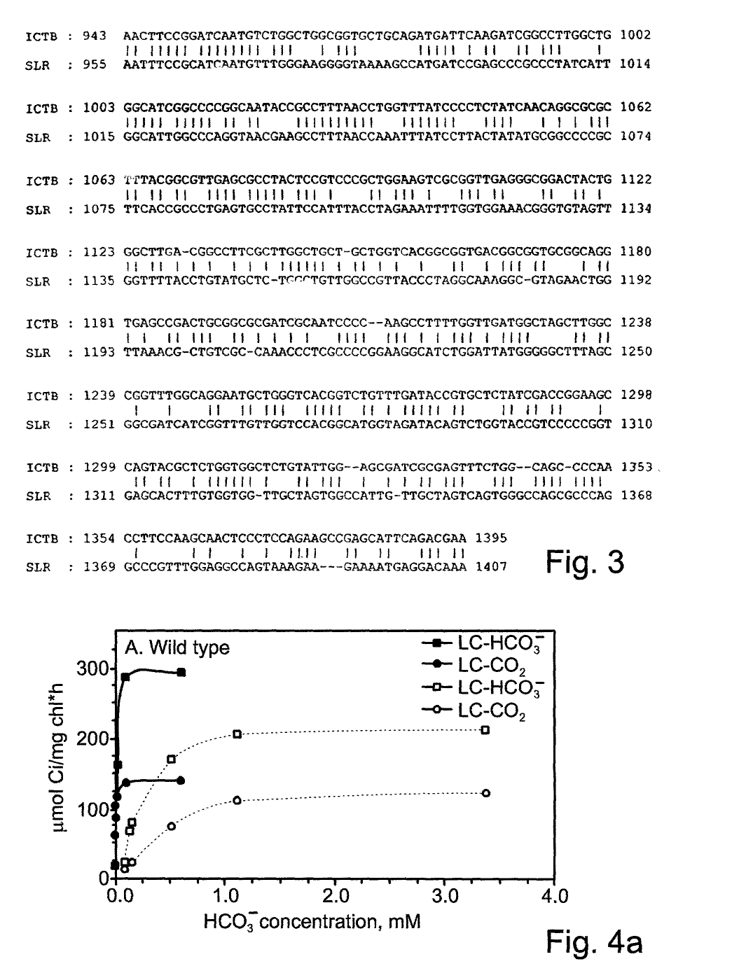

FIGS. 2 and 3 show nucleic and amino acid alignments of ictB and slr1515, the most similar sequence to ictB identified in the gene bank, respectively. Note that the identical nucleotides shared between these nucleic acid sequences (FIG. 2) equal 56%, the identical amino acids shared between these amino acid sequences (FIG. 3) equal 47%, the similar amino acids shared between these amino acid sequences (FIG. 3) equal 16%, bringing the total homology therebetween to 63% (FIG. 3). When analyzed without the transmembrane domains, the identical amino acids shared between these amino acid sequences equal 40%, the similar amino acids shared between these amino acid sequences equal 12%, bringing the total homology therebetween to 52%.

### EXAMPLE 2

### ictB—A Putative Inorganic Carbon Transporter

The protein encoded by ORF467 (SEQ ID NO:3) contains 10 putative transmembrane regions and is a membrane integrated protein. It is somewhat homologous to several oxidation-reduction proteins including the Na⁺/pantothenate symporter of E. Coli (Accession No. P16256). Na⁺ ions are essential for HCO₃⁻ uptake in cyanobacteria and the possible involvement of a Na⁺/HCO₃⁻ symport has been discussed [3, 25, 26]. The sequence of the fourth transmembrane domain contains a region which is similar to the DCCD binding motif in subunit C of ATP synthase with the exception of the two outermost positions, replaced by conservative changes in ORF467. The large number of transport proteins that are homologous to the gene product of ORF467 also suggest that it is also a transport protein, possibly involved in HCO₃⁻ uptake. ORF467 is referred to herein as ictB (for inorganic carbon transport B [27]).

Sequence similarity between cmpA, encoding a 42-kDa polypeptide which accumulates in the cytoplasmic-membrane of low-CO₂-exposed Synechococcus PCC 7942 [28], and nrtA involved in nitrate transport [29], raised the possibility that CmpA may be the periplasmic part of an ABC-type transporter engaged in HCO₃⁻ transport [21, 42]. The role of the 42 kDa polypeptide, however, is not clear since inactivation of cmpA did not affect the ability of Synechococcus PCC7942 [30] and Synechocystis PCC6803 [21] to grow under a normal air level of CO₂ but growth was decreased under 20 ppm CO₂ in air [21]. It is possible that Synechococcus sp. PCC 7942 contains three different HCO₃⁻ carriers: the one encoded by cmpA; IctB; and the one expressed in mutant IL-2 cells exposed to low CO₂ whose identity is yet to be elucidated. These transporters enable the cell to maintain inorganic carbon supply under various environmental conditions.

### EXAMPLE 3

### Transgenic Plants Expressing ictB

The coding region of ictB was cloned downstream of a strong promoter (CaMV 35S) and downstream to, and in frame with, the transit peptide of pea rubisco small subunit. This expression cassette was ligated to vector sequences generating the construct shown in FIG. 6.

Arabidopsis thaliana and Tobacco plants were transformed with the expression cassette described above using the Agrobacterium method. Seedlings of wild type and transgenic Arabidopsis plants were germinated and raised for 10 days under humid conditions. The seedlings were then transferred to pots, each containing one wild type and three transgenic plants. The pots were placed in two growth chambers (Binder, Germany) and grown at 20-21° C., 200 micromol photons m⁻² sec⁻¹ (9h:15h, light:dark). The relative humidity was maintained at 30-35% in one growth chamber and 70-75% in the other. In growth experiments, the plants were harvested from both growth chambers after 18 days of growth. The plants were quickly weighed (fresh weight) and dried in the oven overnight in order to determine the dry weight.

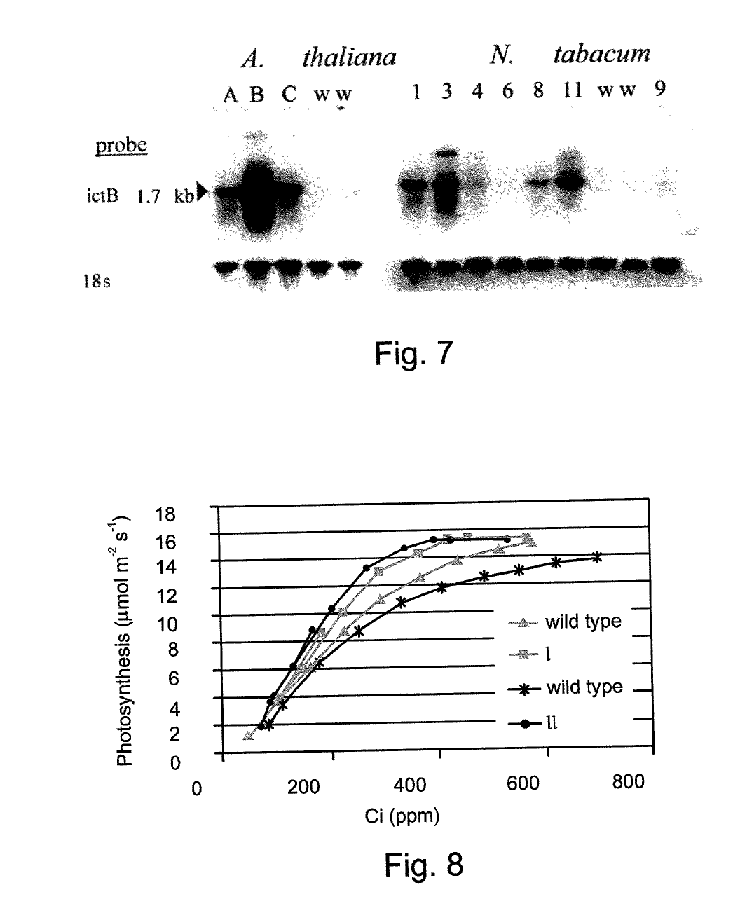

Northern analysis of plant RNA demonstrated that levels of ictB mRNA varied between different transgenic plants, while as expected, ictB mRNA was not detected in the Wild type plants (FIG. 7).

Measurements of the photosynthetic characteristics with respect to CO₂ concentration showed that in both Tobacco (FIG. 8) and Arabidopsis (not shown) the rate of photosynthesis at saturating CO₂ level was similar in the transgenic and wild type plants. On the other hand, under air levels of CO₂ or lower (such as experienced under water stress when the stomata are closed) the transgenic plants exhibited significantly higher photosynthetic rates than the wild type (FIG. 8). Note that the slope of the curve relating photosynthesis to intercellular CO₂ concentration was steeper in the transgenic plants suggesting that the activity of Rubisco was higher in the transgenic plants.

### EXAMPLE 4

### Growth Rate of ictB Transgenic Plants

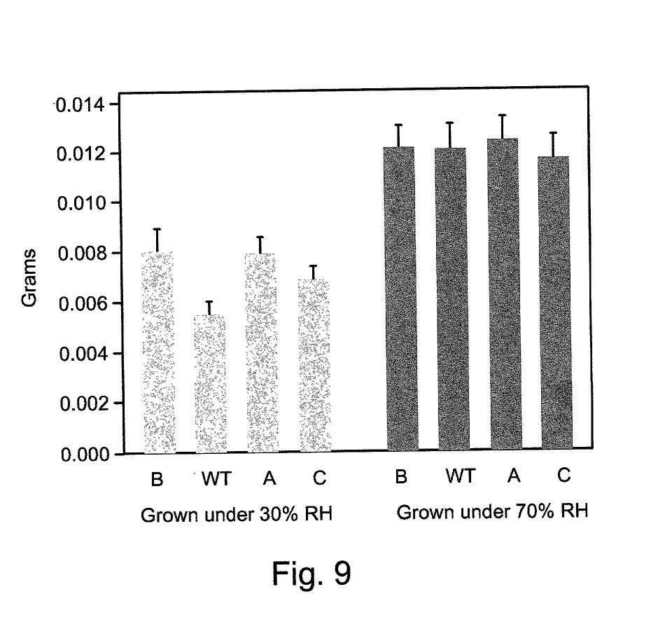

In view of the positive effect of ictB expression on photosynthetic performance, the transgenic plants of the present invention were further tested for growth rates as compared to wild type plants (FIG. 9). Naturally, growth was faster in plants well supplied with water, maintained under the high (above 70%) relative humidity. Under such conditions there was no significant difference between the wild type and the transgenic plants.

On the other hand, the transgenic Arabidopsis plants grew significantly faster than the wild type under conditions of restricted water supply and low (lower than 40%) humidity (FIG. 9). These data demonstrated the potential use of ictB to raise plant productivity particularly under dry conditions where stomatal closure may lead to lower intercellular CO₂ level and thus growth retardation.

The reasons for the very large effect of ictB expression on growth can be due to elevated CO₂ concentration at the site of Rubisco in the transgenic plants, consequent on enhanced HCO₃⁻ entry to the chloroplasts, would be expected to lower the compensation point for CO₂ and to lower the delta ¹³C of the organic matter produced [31]. Table 2 shows that the compensation point was slightly lower in the transgenic plants but the difference was not statistically significant. The slope of the curve relating photosynthesis to intercellular CO₂ concentration (FIG. 8) was steeper in the transgenic plants suggesting (according to accepted models of photosynthesis [31-33]) that the activity of Rubisco was higher than in the wild type. Experiments where we compared the activity of Rubisco in transgenic and wild type plants suggested higher activity in the former (not shown).

TABLE 2
The CO₂ compensation point and the fractionation of stable C
isotopes in wild type and transgenic Arabidopsis and tobacco plants

| | | Compensation |
|---|---|---|
| Sample | d¹³C | point |
| Arabidopsis | | |
| A | 30.8 | 39 ± 4 |
| B | 31.6 | 41 ± 5 |
| WILD TYPE | 31.0 | 46 ± 5 |
| Tobacco | | |
| 3 | 30.8 | 47 ± 6 |
| 11 | 29.9 | 48 ± 7 |
| WILD TYPE | 27.35 | 57 ± 7 |

Thus, applying the teachings of the present invention one can transform plants such as C3 plants including, but not limited to, tomato, soybean, potato, cucumber, cotton, wheat, rice, barley and C4 crop plants, including, but not limited to, corn, sugar cane, sohrgum and others, to thereby generate plants which grow faster, and produce higher crop yield especially under limiting CO₂ and/or water limiting conditions.

### EXAMPLE 5

### ictB homologues

Two additional amino acid sequences exhibiting functional similarity to ictB are listed in Table 3 below. These sequences which encode polypeptides which are 75-80% homologous to ictB (Table 4) can also be used to transform plants in order to achieve the resultant growth or yield enhancement described hereinabove.

TABLE 3

| | Putative/ | | Polynucleotide |
|---|---|---|---|
| | characterized | Protein sequence | sequence |
| Name | function | SEQ ID NO: | SEQ ID NO: |
| Anabaena | none | 6 | 8 |
| PCC7120 | | | |
| Nostoc | none | 7 | 9 |
| punctiforme | | | |

TABLE 4
sequence comparison between ictBand hypothetical amino acid
sequences from Synechocystis sp. PCC 6803, Anabaena PCC7120 and
Nostoc punctiforme

| | | Identical | Similar | Weakly similar | Overall homology |
|---|---|---|---|---|---|
| | Putative/charac. | amino acids | amino acids | amino acids | amino acids |
| Organism | function | % | % | % | % |
| Synechocystis | none | 46.41 | 19.41 | 10.13 | 75.95 |
| slr1515 | | | | | |
| Anabaena | none | 51.37 | 18.32 | 9.68 | 79.37 |
| Nostoc | none | 50.84 | 18.28 | 11.55 | 80.67 |

### Expected Commercial Significance

On the basis of the results obtained with the transgenic Arabidopsis plants (see section 2, above), it is expected that expression of ictB in some of the most important crop plants including: wheat, rice, barley, potato, cotton, soybean, lettuce and tomato will lead to a significant increase in growth and commercial yield especially in regions in which commercial cultivation of food crops is substantially inhibited by growth conditions, such as for example the arid growth conditions characterizing various regions in Africa.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### REFERENCES

1. Kaplan, A., Schwarz, R., Lieman-Hurwitz, J. and Reinhold, L. (1991) Plant Physiol. 97, 851-855.

2. Badger, M. R. and Price, D. G. (1994) Ann. Rev. Plant Physiol. Plant Mol. Biol. 45, 369-399.

3. Kaplan, A., Schwarz, R., Lieman-Hurwitz, J., Ronen-Tarazi, M. and Reinhold, L. (1994) in: The Molecular Biology of the Cyanobacteria (Bryant, D. Ed.), pp. 469-485, Kluwer Academic Pub., Dordrecht, The Netherlands.

4. Ronen-Tarazi, M., Schwarz, R., Bouevitch, A., Lieman-Hurwitz, J., Erez, J. and Kaplan, A. (1995) in: Molecular Ecology of Aquatic Microbes (Joint, I., Ed.), pp. 323-334. Springer-Verlag, Berlin.

5. Kaplan, A., Ronen-Tarazi, M., Zer, H., Schwarz, R., Tchernov, D., Bonfil, D. J., Schatz, D., Vardi, A., Hassidim, M. and Reinhold, L. (1998) Can. J. Bot. 76, 917-924.

6. Sultemeyer, D., Price, G. D., Bryant, D. A. and Badger, M. R. (1997) Planta 201, 36-42.

7. Sultemeyer, D., Klughammer, B., Badger, M. R. and Price, G. D. (1998) Plant Physiol. 116, 183-192.

8. Ronen-Tarazi, M., Shinder, V. and Kaplan, A. (1998) FEMS Microbiol Lett. 159, 317-324.

9. Sambrook, J., Fritsch, E. F. and Maniatis, T. (1989) Molecular Cloning. A Laboratory Manual. Cold Spring Harbor Laboratory Press, Cold Spring Harbor, N.Y.

10. Kaplan, A., Marcus, Y. and Reinhold, L. (1988) in: Methods in Enzymology (Packer, L. and Glazer, A. N., Eds.), pp. 534-539. Academic Press, N.Y.

11. Tchernov, D., Hassidim, M., Luz, B., Sukenik, A., Reinhold, L. and Kaplan, A. (1997) Curr. Biol. 7,723-728.

12. Badger, M. R. and Price, G. D. (1992) Physiol. Plant. 84, 606-615.

13. Ronen-Tarazi, M., Lieman-Hurwitz, J., Gabay, C., Orus, M. and Kaplan, A. (1995) Plant Physiol. 108, 1461-1469.

14. Dolganov, N. and Grossman, A. (1993) J. Bacteriol. 175, 7644-7651.

15. Ronen-Tarazi, M., Bonfil, D. J., Schatz, D. and Kaplan, A. (1998) Can. J. Bot. 76,942-948.

16. Marcus, Y., Schwarz, R., Friedberg, D. and Kaplan, A. (1986) Plant Physiol. 82, 610-612.

17. Schwarz, R., Friedberg, D., Reinhold, L. and Kaplan, A. (1988) Plant Physiol. 88,284-288.

18. Fukuzawa, H., Suzuki, E., Komukai, Y. and Miyachi, S. (1992) Proc. Natl. Acad. Sci. USA 89, 4437-4441.

19. Schwarz, R., Reinhold, L. and Kaplan, A. (1995) Plant Physiol. 108, 183-190.

20. Marco, M., Ohad, N., Schwarz, R., Lieman-Hurwitz, J., Gabay, C. and Kaplan, A. (1993) Plant Physiol. 101, 1047-1053.

21. Ohkawa, H., Sonoda, M., Katoh, H. and Ogawa, T. (1998) in: Proceedings of The Third International Symposium on Inorganic Carbon Uptake by Aquatic Photosynthetic Organisms (Colman, B. Ed.), Can J Bot 76, 1035-1042.

22. Yu, J. W., Price, G. D. and Badger, M. R. (1994) Aust. J. Plant Physiol. 21, 185-195.

23. Kaneko, T., Sato, S., Kotani, H., Tanaka, A., Asamizu, E., Nakamura, Y., Miyajima, N., Hirosawa, M., Sugiura, M., Sasamoto, S., Kimura, T., Hosouchi, T., Matsuno, A., Muraki, A., Nakazaki, N., Naruo, K., Okumura, S., Shimpo, S., Takeuchi, C., Wada, T., Watanabe, A., Yamada, M., Yasuda, M. and Tabata, S. (1996) DNA Res. 3, 109-136.

24. Lieman-Hurwitz J., Schwarz, R., Martinez, F., Maor, Z., Reinhold, L., and Kaplan, A. (1990) in: Proceedings of The Second International Symposium on Inorganic Carbon Uptake by Aquatic Photosynthetic Organisms (Colnan, B. Ed.), Can J Bot 69:945-950.

25. Kaplan, A., Schwarz, R., Ariel, R., and Reinhold, L. (1990) in: Regulation of Photosynthetic Processes (Kanay, R., Katoh, R. S. and Miyachi, S. Eds.), Special Issue of the Botanical Magazine, vol. 2, pp. 53-71, Tokyo.

26. Tyrrell, P. N., Kandasamy, R. A., Crotty, C. M. and Espie, G. S. (1996) Plant Physiol. 112, 79-88

27. Ogawa, T. (1991) Plant Physiol. 96, 280-284.

28. Omata, T. and Ogawa, T. (1986) Plant Physiol. 80, 525-530.

29. Omata, T. (1992) in: Research in Photosynthesis (Murata, N. Ed.) vol. III, 807-810, Kluwer Academic Pub., Dodrecht, The Netherlands.

30. Omata, T., Carlson, T. J., Ogawa, T. and Pierce, J. (1990) Plant Physiol. 93, 305-311.

31. Woodrow, I. E. and Berry, J. A. (1988) Ann. Rev. Plant Physiol 39, 533-594.

32. Mott, K. A. and Woodrow, I. E. (2000) Journal of Experimental Botany 51,399-406.

33. Poolman, M. G., Fell, D. A. and Thomas, S. (2000) Journal of Experimental Botany 51, 319-328.

34. Kaplan, A. and Reinhold, L. (1999) Annu. Rev. Plant Physiol. Plant Mol. Biol. 50, 539-570.

35. Spreitzer, R. J. (1999) Photosynthesis Research 60, 29-42.

36. Horton, P. (2000) Journal of Experimental Botany 51, 475-485.

37. Matsuoka, M., Nomura, M., Agarie, S., Miyao-Tokutomi, M. and Ku, M. S. B. (1998) Journal of Plant Research 111, 333-337.

38. Nomura, M. et al. (2000) Plant Mol. Biol. 44, 99-106.

39. Takeuchi, K., Akagi, H., Kamasawa, N., Osumi, M. and Honda, H. (2000) Planta 211, 265-274.

40. Suzuki, S., Murai, N., Bumell, J. N. and Arai, M. (2000) Plant Physiol. 124, 163-172.

41. Bonfil, D. J., Ronen-Tarazi, M., Sultemeyer, D., Lieman-Hurwitz, J., Schatz, D. and Kaplan, A. (1998) FEBS Lett. 430, 236-240.

42. Maeda, S-I., Price, G. D., Badger, M. R., Enomoto, C. and Omata, T. (2000) J. Biol. Chem. 275, 20551-20555

What is claimed is:

1. A method of enhancing growth and/or commercial yield of a plant, the method comprising expressing within the plant a polypeptide including an amino acid sequence at least 60% homologous to that set forth in SEQ ID NOs:3, 5, 6 or 7.

2. The method of claim 1, wherein the plant is grown in an environment characterized by humidity lower than 40%.

3. The method of claim 1, wherein the plant is grown in an environment characterized by an intercellular CO₂ concentration lower then 10 micromolar.

4. The method of claim 1, wherein expressing said polypeptide within the plant is effected by transforming at least a portion of the plant cells with a nucleic acid construct including a polynucleotide region encoding said polypeptide.

5. The method of claim 4, wherein said transforming is effected by a method selected from the group consisting of agrobacterium mediated transformation, viral infection, electroporation and particle bombardment.

6. The method of claim 1, wherein said amino acid sequence is as set forth by SEQ ID NOs:3, 5, 6 or 7.

7. The method of claim 4, wherein said nucleic acid construct further includes a second polynucleotide region encoding a transit peptide.

8. The method of claim 4, wherein said nucleic acid construct further includes a promoter sequence for directing transcription of said first polynucleotide region.

9. The method of claim 4, wherein said nucleic acid construct further includes a promoter sequence for directing transcription of said first and said second polynucleotide regions.

10. The method of claim 8, wherein said promoter is functional in eukaryotic cells.

11. The method of claim 10, wherein said promoter is selected from the group consisting of a constitutive promoter, an inducible promoter, a developmentally regulated promoter and a tissue specific promoter.

12. The method of claim 1, wherein said plant is a C3 plant.

13. The method of claim 12, wherein said C3 plant is selected from the group consisting of tomato, soybean, potato, cucumber, cotton, wheat, rice, barley, lettuce, solidago, banana and poplar.

14. The method of claim 1, wherein said plant is a C4 plant.

15. The method of claim 14, wherein said C4 plant is selected from the group consisting of corn, sugar cane, sohrgum.

16. The method of claim 1, wherein the plant expressing said polypeptide is characterized by a growth rate which is at least 10% higher than that of a similar plant not expressing said polypeptide when both are grown under similar growth conditions.

17. The method of claim 16, wherein said growth rate is determined by at least one growth parameter selected from the group consisting of increased fresh weight, increased dry weight, increased root growth, increased shoot growth and increased flower development over time.

18. A transformed plant expressing a polypeptide including an amino acid sequence at least 60% homologous to that set forth in SEQ ID NOs:3, 5, 6 or 7, said transformed plant characterized by an enhanced growth as compared to similar non transformed plant grown under similar growth conditions.

19. The transformed plant of claim 18, wherein said growth conditions include humidity conditions of less than 40%.

20. The transformed plant of claim 18, wherein said amino acid sequence is as set forth by SEQ ID NOs:3, 5, 6 or 7.

21. The transformed plant of claim 18, wherein said transformed plant is a C3 plant.

22. The transformed plant of claim 21, wherein said C3 plant is selected from the group consisting of tomato, soybean, potato, cucumber, cotton, wheat, rice, barley, lettuce, solidago, banana, poplar, citrus.

23. The transformed plant of claim 18, wherein said transformed plant is a C4 plant.

24. The transformed plant of claim 23, wherein said C4 plant is selected from the group consisting of corn, sugar cane, sohrgum.

25. The transformed plant of claim 18, wherein a growth rate of said transformed plant is at least 10% higher than that of a similar non transformed plant when both are grown under similar growth conditions.

26. The transformed plant of claim 23, wherein said growth rate is determined by at least one growth parameter selected from the group consisting of fresh weight, dry weight, root growth, shoot growth and flower development.

27. The transformed plant of claim 18, wherein said transformed plant is further characterized by an increased commercial yield as compared to similar non transformed plant grown under similar conditions.

28. The transformed plant of claim 18, wherein said growth conditions include water stress, low humidity, salt stress, and/or low CO₂ conditions.

29. A nucleic acid expression construct comprising:
(a) a first polynucleotide region encoding a polypeptide including an amino acid sequence at least 60% homologous to that set forth by SEQ ID NOs:3, 5, 6 or 7; and
(b) a second polynucleotide region functional as a promoter and being for directing the transcription of said first polynucleotide region in eukaryotic cells.

30. The nucleic acid expression construct of claim 29, wherein said promoter is selected from the group consisting of a constitutive promoter, an inducible promoter, a developementally regulated promoter and a tissue specific promoter.

31. The nucleic acid expression construct of claim 29, wherein said promoter is a plant promoter.

32. The nucleic acid expression construct of claim 29, wherein said first polynucleotide region further encodes a transit peptide being translationally fused to said polypeptide.
